(19) United States
(12) Patent Application Publication
(10) Pub. No.: US 2025/0082803 A1
(43) Pub. Date: Mar. 13, 2025

(54) HYDROGEN PEROXIDE VAPORIZATION AND DECOMPOSITION APPARATUS

(71) Applicant: WOOJUNG BIO, INC., Hwaseong-si, Gyeonggi-do (KR)

(72) Inventors: Byung Nyun CHUN, Suwon-si, Gyeonggi-do (KR); Yun Jae JUNG, Osan-si, Gyeonggi-do (KR); Joo Hyung CHUN, Suwon-si, Gyeonggi-do (KR)

(21) Appl. No.: 18/284,390

(22) PCT Filed: Sep. 21, 2023
(86) PCT No.: PCT/KR2023/014349

§ 371 Date: Sep. 27, 2023

(30) Foreign Application Priority Data
Dec. 15, 2022 (KR) 10-2022-0175659

(51) Int. Cl. A61L 2/18; A61L 2/22

(52) U.S. Cl. CPC A61L 2/186; A61L 2/22; A61L 2202/11; A61L 2202/13

(57) ABSTRACT

A hydrogen peroxide vaporization and decomposition apparatus includes a housing, a vaporizer provided on one side of the inside of the housing so as to vaporize a hydrogen peroxide solution, a vapor decomposition device provided in the vaporization part on one side of the inside of the housing so as to decompose hydrogen peroxide vapor, and a diffusion fan provided on an upper side of the housing so as to diffuse hydrogen peroxide vapor vaporized in the vaporizer or air decomposed in the vapor decomposition device.

### TECHNICAL FIELD

The present invention relates to a hydrogen peroxide vaporization and decomposition apparatus and, more specifically, to a hydrogen peroxide vaporization and decomposition apparatus, which can maximize the vaporization efficiency of a hydrogen peroxide solution in a simple structure, thereby performing efficient sterilization, and can simultaneously perform decomposition of hydrogen peroxide vapor diffused into a target space through a vapor decomposition device provided together in a single housing.

### BACKGROUND ART

A chemical sterilizer is an apparatus for performing a sterilization process using a sterilant such as hydrogen peroxide gas, ethylene oxide gas, chlorine dioxide gas, and the like.

In a conventional chemical sterilizer using hydrogen peroxide as a sterilant, the hydrogen peroxide can be vaporized in a sterilization chamber at low pressure of 3 torr or less. The vaporized sterilant is supplied to the interior of the chamber having a given volume to perform sterilization, and in this case, an amount of sterilant supplied is diffused at a pressure greater than or equal to 20 torr so that the sterilant is sufficiently supplied, while being kept in a state of gas at a predetermined temperature. Like this, only when the sterilant is kept to the state of gas, it is transferred to a sterilization article located in the interior of the chamber and the inside of the sterilization article, thereby performing successful sterilization.

A conventional plasma sterilizer generally has several liters of a sterilization chamber, and an amount of sterilant (hydrogen peroxide solution) supplied at a temperature of about 60° C. is several milliliters. The larger the capacity of sterilization chamber is, the larger the amount of hydrogen peroxide supplied is, so that an amount of sterilant to be vaporized in a vaporizer becomes increased. Therefore, there is a need to develop a vaporizer for allowing a hydrogen peroxide solution to have sufficient residence time therein and effectively heating and vaporizing the hydrogen peroxide solution.

### PRIOR ART DOCUMENT

### Patent Document

(Patent Document 1) KR 10-0867654

### DISCLOSURE OF THE INVENTION

### Technical Problem

Accordingly, the present invention has been made in view of the above-mentioned problems occurring in the related art, and it is an object of the present invention to provide a hydrogen peroxide vaporization and decomposition apparatus, which can maximize the vaporization efficiency of a hydrogen peroxide solution in a simple structure, thereby performing efficient sterilization, and can simultaneously decompose hydrogen peroxide vapor diffused into a target space through a vapor decomposition device provided in a single housing.

The object of the present invention is not limited thereto, and other objects not mentioned herein will be obviously understood by those skilled in the art through the following description.

### Technical Solution

To accomplish the above-mentioned objects, according to the present invention, there is provided a hydrogen peroxide vaporization and decomposition apparatus includes a housing, a vaporizer provided on one side of the inside of the housing so as to vaporize a hydrogen peroxide solution, a vapor decomposition device provided in the vaporization part on one side of the inside of the housing so as to decompose hydrogen peroxide vapor, a diffusion fan provided on an upper side of the housing so as to diffuse hydrogen peroxide vapor vaporized in the vaporizer or air decomposed in the vapor decomposition device, wherein the vaporizer includes a body part having the shape of a hollow column, a vaporization part disposed on one side of the inside of the body part and having a predetermined capacity to accommodate the hydrogen peroxide solution, support parts for supporting the vaporization part to allow the vaporization part to be spaced apart from the inside of the body part by a predetermined distance, a heating part disposed on one side of the vaporization part to heat the vaporization part, a discharge part disposed on top of the body part to induce the hydrogen peroxide vapor generated from the vaporization part to be discharged, a discharge fan provided so as to be disposed on one side of the inside of the body part at a position relatively lower than that of the vaporization part, thereby improving discharge efficiency of the hydrogen peroxide vapor that is vaporized in the vaporization part and discharged through the discharge part, and a condensate tray provided between the vaporization part and the discharge fan so as to accommodate condensate formed from at least one of the vaporization part, the heating part, the discharge part, and the diffusion fan.

Herein, the vapor decomposition device is provided at a position relatively lower than that of the vaporizer.

In addition, the vapor decomposition device and the vaporizer are provided so that central axes thereof are offset from each other.

In addition, the diffusion fan is provided so as to be spaced apart from an upper end of the discharge part at an interval in the range of 10 mm to 150 mm.

In addition, the vaporization part is configured so that a distance from a vapor generation surface of the vaporization part to the upper end of the discharge part has a height in the range of 50 mm to 300 mm.

In addition, the upper end area of the discharge part is formed to be 30% to 50% of an introduction portion area of the diffusion fan into which the hydrogen peroxide vapor or the air decomposed in the vapor decomposition device is introduced.

### Advantageous Effects of the Invention

According to the present invention, the hydrogen peroxide vaporization and decomposition apparatus has the effects as follows.

First, the hydrogen peroxide vaporizer is simple in configuration and maximizes the vaporization efficiency of the hydrogen peroxide solution supplied to the vaporization part through the configuration of the vaporization part.

Second, the vaporization part has wall surfaces of a predetermined height so that the hydrogen peroxide solution supplied to the vaporization part bounces up from the bottom of the vaporization part and comes into contact with the wall surfaces of the vaporization part, thereby further improving the vaporization efficiency of the hydrogen peroxide solution.

Third, owing to protrusions or partition walls of the vaporization part, a contact area between the vaporization part and the hydrogen peroxide solution becomes increased so that sufficient vaporization efficiency of the hydrogen peroxide solution can be obtained while reducing the volume of the vaporization part as well as the volume of the hydrogen peroxide vaporizer can be reduced.

Fourth, the area of the vaporization part can be reduced as described above and accordingly the volume of the hydrogen peroxide vaporizer as well as the entire volume of a sterilizer can be reduced.

Fifth, through the optimized configuration of the discharge part, the hydrogen peroxide vapor is prevented from flowing back downward in the discharge part or resistance in air flow is minimized. In addition, a capacity ratio between the discharge fan of the discharge part and the diffusion fan and the separation distance between the discharge part and the diffusion fan optimized, thereby maximizing the diffusion efficiency of the hydrogen peroxide vapor.

Sixth, as described above, the vaporization and diffusion efficiency of the hydrogen peroxide solution are maximized, thereby maximizing sterilization efficiency.

Seventh, since the vaporization part is fixed to the body part with minimum contact points through the support parts, heat loss that occurs when heat supplied from the heating part comes into contact with external air through the body part is minimized, thereby maximizing heating efficiency. Furthermore, since the vaporization part is provided to be spaced apart from the body part through the support parts, the vaporization part is structured to be separated from combustion materials, thereby ensuring the safety of use even if a fire occurs in the vaporization part during use.

Eighth, since the generated hydrogen peroxide vapor is discharged while being mixed with vapor of high temperature supplied by the discharge fan, the hydrogen peroxide vapor can be prevented from being cold and liquefied again during movement so as to be stably diffused up to a target point.

Ninth, the hydrogen peroxide vaporizer is entirely made of an aluminum material, which is a highly chemical-resistant and lightweight material, thereby minimizing the weight of the hydrogen peroxide vaporizer and the entire device.

Tenth, even if condensate forms depending on use environment or the like despite the optimized structure, device failure due to short circuits or the like can be prevented through inclination structure of the discharge part and the condensate tray provided inside the body part.

Eleventh, the vaporizer and the vapor decomposition device are integrated through the housing so that hydrogen peroxide vapor diffused in a target space can be decomposed without providing any separate decomposition device, thereby maximizing convenience of use.

Twelfth, the vaporizer and the vapor decomposition device are arranged up and down with central axes thereof offset from each other so as to prevent decomposed air decomposed in the vapor decomposition device from being smoothly discharged to the outside and increase the time for the hydrogen peroxide vapor to stay in the vapor decomposition device, thereby maximizing the decomposition efficiency of hydrogen peroxide vapor.

The effects of the present invention are not limited to the above-mentioned effects, and other effects not mentioned will be clearly understood by those skilled in the art from the claims.

### BRIEF DESCRIPTION OF DRAWINGS

The following drawings herein illustrate preferred embodiments of the present invention and serve to facilitate the general understanding of the scope of the present invention, together with the detailed description of the present invention. Therefore, the present invention should not be construed as limited to the matters described in such drawings.

FIG. 1 is a perspective view of a hydrogen peroxide vaporization and decomposition apparatus according to the present invention;

FIG. 2 is a front view showing the inside of the hydrogen peroxide vaporization and decomposition apparatus according to the present invention;

FIG. 3 is a side view showing the inside of the hydrogen peroxide vaporization and decomposition apparatus according to the present invention;

FIG. 4 is a longitudinal sectional view of a vaporizer according to the present invention;

FIG. 5 is a cross-sectional view of a body part of the vaporizer according to the present invention;

FIG. 6 is a cross-sectional view of the vaporizer according to the present invention, showing flows of fluid when vaporizing hydrogen peroxide vapor in the vaporizer; and

FIG. 7 is a longitudinal sectional view of the hydrogen peroxide vaporization and decomposition apparatus according to the present invention, showing flows of fluid according to the operation of the vapor decomposition device.

### MODE FOR INVENTION

Objects, characteristics and advantages of the present invention will be more clearly understood from the detailed description as will be described below and the attached drawings. Before the present invention is disclosed and described, it is to be understood that the disclosed embodiments are merely exemplary of the invention, which can be embodied in various forms. Therefore, specific structural and functional details disclosed herein are not to be interpreted as limiting, but merely as a basis for the claims and as a representative basis for teaching one of ordinary skill in the art to variously employ the present invention in virtually any appropriately detailed structure.

Terms used in this application are used to only describe specific exemplary embodiments and are not intended to restrict the present invention. An expression referencing a singular value additionally refers to a corresponding expression of the plural number, unless explicitly limited otherwise by the context. In the description, when it is said that one portion is described as “comprises” and/or “comprising” any component, one element further may include other components unless no specific description is suggested. In the description, the same reference numerals will be used to describe the same components. It shall also be understood that the terms “and/or” used herein are intended to signify and include any or all possible combinations of one or more of the associated listed items, unless the context clearly indicates otherwise. It shall be understood that, although the terms “first,” “second,” etc. may be used herein to describe various components, the components should not be limited by these terms. These terms are only used to distinguish one component from another. For example, without departing from the scope of the present disclosure, a first component may be termed as a second component.

All terms used herein, including technical or scientific terms, unless otherwise defined, have the same meanings which are typically understood by those having ordinary skill in the art. The terms, such as ones defined in common dictionaries, should be interpreted as having the same meanings as terms in the context of pertinent technology, and should not be interpreted as having ideal or excessively formal meanings unless clearly defined in the specification.

Hereinafter, a preferred embodiment of the present invention will be described in detail with reference to the accompanying drawings.

### Configuration of Hydrogen Peroxide Vaporization and Decomposition Apparatus

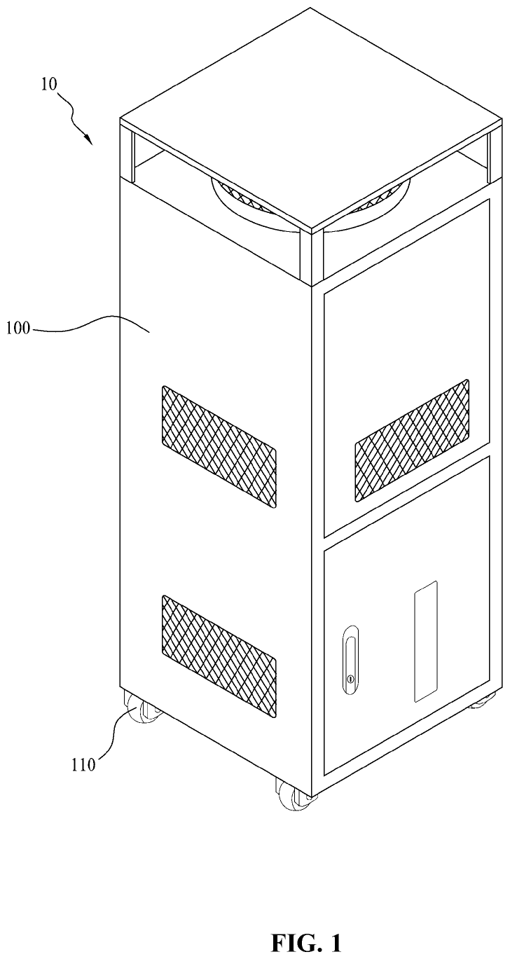

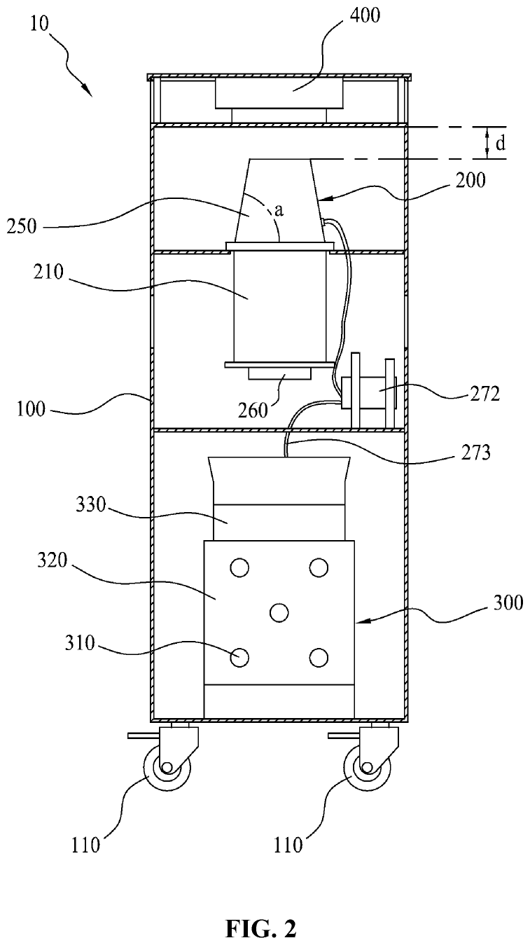

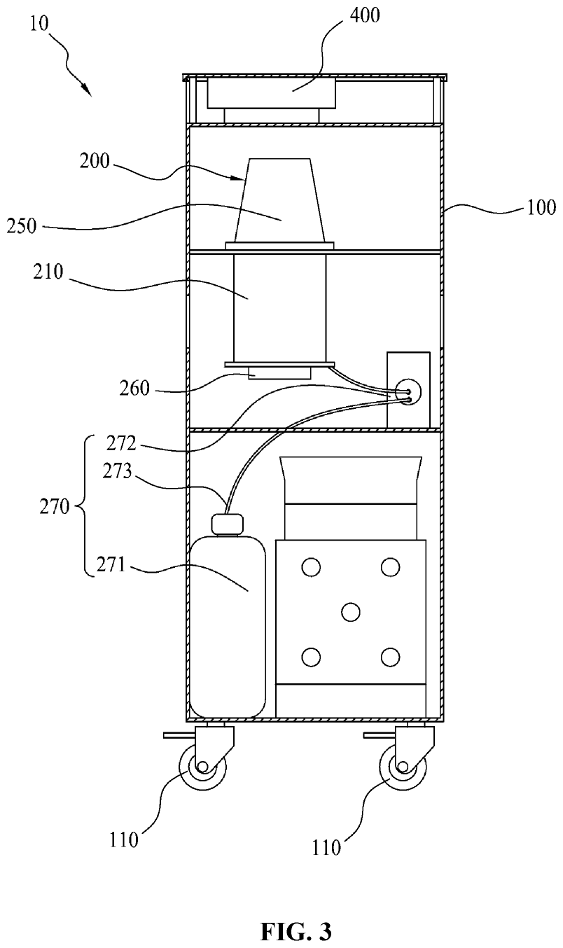

FIG. 1 is a perspective view of a hydrogen peroxide vaporization and decomposition apparatus according to the present invention, FIG. 2 is a front view showing the inside of the hydrogen peroxide vaporization and decomposition apparatus according to the present invention, and FIG. 3 is a side view showing the inside of the hydrogen peroxide vaporization and decomposition apparatus according to the present invention. A hydrogen peroxide vaporization and decomposition apparatus 10 according to the present invention is an apparatus, which can sterilize the inside of a sterilization space by using hydrogen peroxide vapor and decompose the hydrogen peroxide vapor distributed inside the sterilization space, after sterilization is completed, by using a single housing. This hydrogen peroxide vaporization and decomposition apparatus 10 mainly includes, as shown in FIG. 1 to FIG. 3, a housing 100, a vaporizer 200, a vapor decomposition device 300, and a diffusion fan 400.

The housing 100 forms the exterior of the hydrogen peroxide vaporization and decomposition apparatus 10 according to the present invention, and can be made of various sizes and shapes in consideration of a sterilization space area, a hydrogen peroxide solution capacity, or the like. This housing 100 is formed hollow so that the vaporizer 200 and the vapor decomposition device 300 can be provided therein. This housing 100 may include partition walls, support pipes or fixing protrusions which protrude in the horizontal direction so that the vaporizer 200 and the vapor decomposition device 300 can be respectively provided at upper and lower portions inside, wherein such structures are preferably provided allowing air flow. In addition, a top surface of the housing 100 or one side of an upper portion of a side wall may be provided with the diffusion fan 400 or a through hole so as to allow air to flow to the diffusion fan 400. Furthermore, depending on usage, at least one or more through holes may be formed to allow external air to flow into the housing 100.

Additionally, wheels 110 may be further provided on one side of a lower surface of the housing 100 so as to easily move the housing 100.

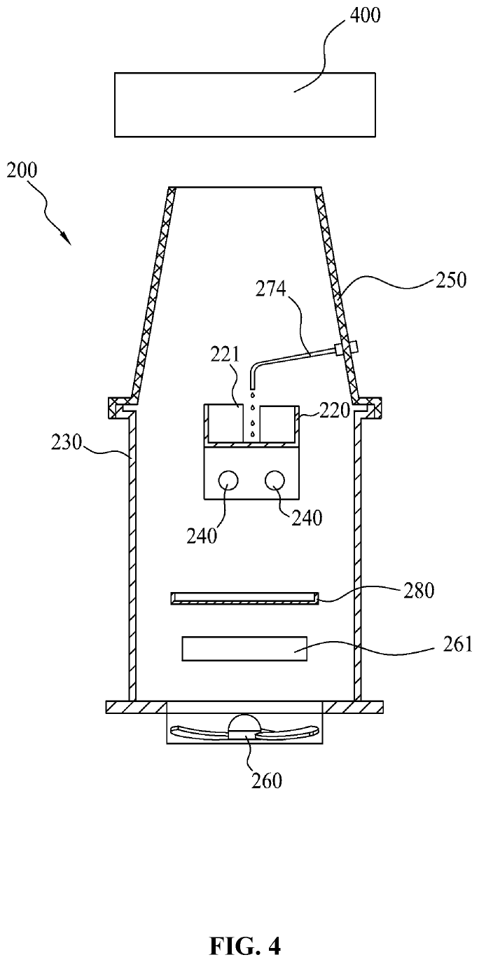

FIG. 4 is a longitudinal sectional view of the vaporizer according to the present invention. The vaporizer 200 is a device that is provided on one side of the housing 100, preferably, on one side of an upper portion in the housing 100, and generates hydrogen peroxide vapor to sterilize the inside of a target space. The vaporizer 200 may be any device that generates hydrogen peroxide vapor by using a hydrogen peroxide solution. In an embodiment, the vaporizer 200 includes, as shown in FIG. 4, a body part 210, a vaporization part 220, support parts 230, a heating part 240, a discharge part 250, a discharge fan 260, a condensate tray 280 and a solution supply part 270.

The body part 210 forms an outer appearance of the vaporizer 200 and provides spaces for securing each device constituting the vaporizer 200. The body part 210 may be made in various sizes in consideration of a target space area, a hydrogen peroxide vapor capacity depending on the target space area, or the like. This body part 210 may have any shape as long as it is a hollow device so that the above described components can be combined inside or outside. Preferably, the body part 210 has the shape of a hollow tube, column, or pipe, of which top and bottom surfaces are open. According to an embodiment, the body part 210 is circular in cross section. However, the body part 210 may have various shapes such as polygons, irregular shapes or the like depending on usage.

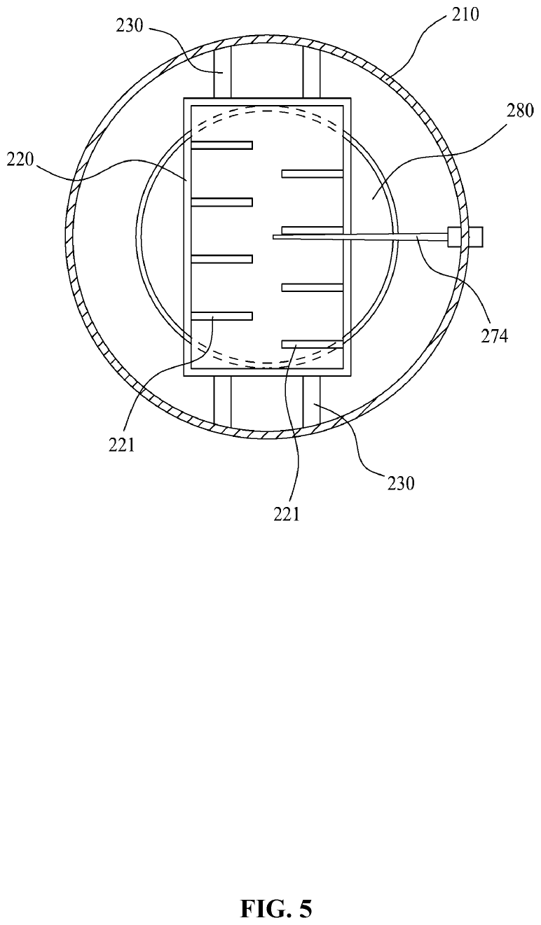

FIG. 5 is a cross-sectional view of the body part of the vaporizer according to the present invention. The vaporization part 220 is a device provided on one side of the inside of the body part 210 so as to accommodate and vaporize the hydrogen peroxide solution introduced from the outside. The vaporization part 220 is disposed on one side in the body part 210, preferably, on one side of a lower portion in the body part 210 and spaced apart from inner walls of the body part 210 through the support parts 230. Herein, the vaporization part 220 can be any device as long as it has wall surfaces formed with a predetermined height on edge portions so that grooves are formed inside so as to accommodate a predetermined capacity of a supplied hydrogen peroxide solution. However, the vaporization part 220 is preferably formed in a hexahedral shape so that heat transferred from the heating part 240 is uniformly transferred to the vaporization part 220 on the whole. Herein, the vaporization part 220 has a size, in which a hydrogen peroxide solution can be vaporized at a minimum of about 1 g to a maximum of about 22 g per minute and, preferably, about 8 g to about 20 g per minute. More preferably, the vaporization part 220 has a size so that even if vaporization of the hydrogen peroxide solution is delayed for a predetermined time, the hydrogen peroxide solution does not overflow from the vaporization part 220. To this end, the vaporization part 220 is preferably configured to have a width of 50 to 60 mm, a length of 80 to 100 mm, and a height of about 50 mm and, more preferably, a width of about 55 mm, a length of about 90 mm, and a height of about 40 mm. If the vaporization part 220 has a height less than 30 mm, the hydrogen peroxide solution supplied from the solution supply part 270 may overflow from the vaporization part 220 or splashes out of the vaporization part 220 due to drop repulsion. Further, if the vaporization part 220 has a height greater than 50 mm, temperature transferred from the heating part 240 is not transferred smoothly to the wall surfaces of the vaporization part 220, so that temperature change of the wall surfaces of the vaporization part 220 may significantly increase, failing to achieve uniform vaporization of the hydrogen peroxide solution.

Further, the vaporization part 220 is made of a high chemical resistant material, preferably, an aluminum material so as to be prevented from being corroded by the hydrogen peroxide solution. According to the present invention, for the convenience of the explanation, the vaporization part 220 has the shape of a hollow hexahedron whose top is open, but is not limited thereto. Herein, the vaporization part 220 may be further provided with protrusions or partition walls 221 on one side of at least one of the inner bottom or wall surfaces thereof so as to increase a contact area with respect to the hydrogen peroxide solution accommodated therein, thereby improving vaporization efficiency. Accordingly, since the contact area with the hydrogen peroxide solution increases due to the protrusions or partition walls 221, the volume of the vaporization part 220 may be reduced, thereby reducing the entire volume of the hydrogen peroxide vapor vaporizer 200, while achieving sufficient vaporization efficiency.

As mentioned above, the support parts 230 serve to stably support the vaporization part 220 on one side of the inside of the body part 210. Such a support part 230 may be provided in any form as long as it is a device capable of stably fixing and supporting the vaporization part 220 inside the body part 210. However, the support parts 230 are preferably provided to have minimum contact points so as to maintain a state in which the body part 210 and the vaporization part 220 are spaced apart from each other, thereby facilitating smooth flow of air generated by the discharge fan 260, which will be described later, and minimizing heat loss by minimizing contact with the body part 210. For example, it is preferable that two or more bar-shaped support parts 230 are spaced apart from each other so as to form passages through which air can flow. It is also possible to provide only one support part 300, if the support part 230 has a structure capable of sufficiently and stably supporting the evaporation part 220. However, since it is not easy to acquire sufficiently stable support with just one support part 230, it is preferable to provide at least two or more bar-shaped support parts 230, wherein a separation distance between the support parts 230 may be set to various lengths in consideration of the capacity of a sterilizer, the vaporization efficiency and heat loss rate of the vaporization part 220, or the like.

The heating part 240 is connected to one side of the vaporization part 220, generates heat for vaporizing the hydrogen peroxide solution accommodated in the vaporization part 220, and transfers the heat to the vaporization part 220. Such a heating part 240 may be any heating device that can heat the vaporization part 220 to a temperature greater than or equal to the evaporation point of the hydrogen peroxide solution. In one embodiment, the heating part 240 may adopt a heater rod, which is inexpensive, has a simple structure and high efficiency, and is ready to control. Herein, it is preferable to use two heater rods of 1000 W so as to provide a heating device of 2000 W. In order to measure evaporation efficiency according to the capacity of the heating part 240, measurement was performed at a capacity of less than 2,000 W, while changing the capacity to 500 W, 1,000 W and 1,500 W for the heating part 240. As a result, there is a problem in that it is difficult to maintain the temperature of the vaporization part 220 constant. That is, the temperature of the vaporization part 220 varies in a wide range due to such a small capacity and is not uniformly maintained due to a drastic temperature change and, as a result, the vaporization efficiency of the hydrogen peroxide solution is not maintained constant.

The heating part 240 preferably maintains the temperature of the vaporization part 220 at about 130° C. to 150° C., and more preferably about 140° C. The results of comparison between the temperature change of the vaporization part 220 and the vaporization efficiency of the hydrogen peroxide solution are shown in [Table 1] below. Herein, the vaporization part 220 has a size of 54 mm in width, 88 mm in length, and 40 mm in height and can accommodate a maximum of about 24 ml of hydrogen peroxide solution. An experiment was conducted by supplying about 20 ml of the hydrogen peroxide solution per minute to the vaporization part 220 and measuring the amount of hydrogen peroxide solution vaporized for one minute.

TABLE 1

| Temperature | Amount of vaporized hydrogen |
|---|---|
| (° C.) | peroxide solution (g) |
| 120 | 10 |
| 130 | 14 |
| 140 | 16 |
| 150 | 14 |

As shown in table 1, it could be checked that the vaporization efficiency of the hydrogen peroxide solution increased in the process of increasing the temperature of the vaporization part 220 to 140° C. but the vaporization efficiency decreased at temperature above 150° C. This is because the evaporation point of the hydrogen peroxide solution is about 140° C. It was confirmed that at 150° C., the hydrogen peroxide solution supplied to the vaporization part 220 began to be vaporized and was exploded to the outside of the vaporization part 220, resulting in a risk of an accident. In addition, the hydrogen peroxide solution was not vaporized well and splashed to the outside of the vaporization part 220. Consequently, the vaporization efficiency was reduced. In addition, if the temperature of the vaporization part 220 is less than or equal to 120° C., the vaporization efficiency of the hydrogen peroxide solution may decrease. Further, since the amount of hydrogen peroxide solution supplied becomes greater than the amount of hydrogen peroxide solution vaporized, the temperature of the vaporization part 220 may decrease over time or the hydrogen peroxide solution may overflow out of the vaporization part 220. Therefore, it can be appreciated that a maximum vaporization efficiency can be obtained stably by maintaining the vaporization part 220 at a temperature of about 130° C. to 150° C., preferably about 140° C. by means of the heating part 240. To this end, it is preferable to use a heating part 240 formed of two heater rods each having a capacity of about 1,000 W. The discharge part 250 is provided to the open top portion of the body part 210 so as to prevent the hydrogen peroxide vapor vaporized by the vaporization part 220 from spreading arbitrarily, thereby preventing a decrease in diffusion efficiency. In addition, the discharge part 250 induces a discharge direction of the hydrogen peroxide vapor so that the hydrogen peroxide vapor can be smoothly discharged to the diffusion fan 400 that is provided above the housing 100 so as to ensure smooth diffusion. The discharge part 250 is preferably tapered upward so as to be gradually narrower toward the top, so that the hydrogen peroxide vapor generated in the vaporization part 220 moves up and gathers in the center thereof, discharge speed of the hydrogen peroxide vapor is increased through air jet effect, and condensate formed by and falling from the diffusion fan 400 depending on the surrounding environment is guided to flow along the outer surface of the discharge part 250, thereby preventing the condensate from flowing into the discharge part 250 as much as possible. That is, a diameter of a lower end of the discharge part 250 corresponds to a diameter of an upper end of the body part 210 and becomes gradually reduced upward. The discharge part 250, of which diameter gradually decreases upward, may be formed inclined at an inclination angle a in the range of 60° to 85°, wherein the inclination angle is preferably in the range of 75° to 85°, and more preferably about 80°.

Herein, it is preferable that a diameter of an upper end of the discharge part 250 is 30% to 50% of an inlet diameter of the diffusion fan 400 so that the vapor discharged from the discharge part 250 is introduced into the diffusion fan 400 without any loss. Further, the discharge part 250 preferably has a length that allows the hydrogen peroxide vapor generated from the vaporization part 220 to be quickly discharged to the outside before it turns into water droplets, and the discharge part 250 may be adjusted to various lengths depending on the capacity of the vaporization part 220 or the like. Preferably, a distance d from a vapor generation surface of the vaporization part 220 to the upper end of the discharge part 250 is in the range of about 50 mm to 300 mm, more preferably about 100 mm to 250 mm, and even more preferably 150 mm to 200 mm. If the distance d from the vapor generation surface of the vaporization part 220 to the upper end of the discharge part 250 is shorter than 50 mm, air introduced through the discharge fan 260 and heated through an air heater 261 may not be mixed smoothly with the hydrogen peroxide vapor. Further, if the distance d from the vapor generation surface of the vaporization part 220 to the upper end of the discharge part 250 exceeds 300 mm, the heated air and the hydrogen peroxide vapor which are mixed together rise and temperature thereof decreases. Therefore, the heated air and the hydrogen peroxide vapor which are mixed together may not flow smoothly into the diffusion fan 400 or may condense and thus form condensate.

That is, the inclination angle α of the discharge part 250 can be selectively determined within an angle range of 60° to 85° by comprehensively calculating the ratio between the inlet diameter of the diffusion fan 400 and the diameter of the upper end of the discharge part 250 and the distance d from the vapor generation surface of the vaporization part 220 to the upper end of the discharge part 250, as described above, and considering position where the condensate formed by the diffusion fan 400 is generated, or the like. Further, the discharge part 250 preferably has a cylindrical sectional shape so that vapor of high temperature can be prevented from flowing back to the bottom due to convection or air flow resistance such as whirlwind generation can be minimized, thereby allowing the vapor to quickly flow into the diffusion fan 400. If the discharge part 250 has a circular cross-section, the discharge part 250 achieves efficient temperature control more than a discharge part having a polygonal cross-section in many ways.

The discharge fan 260 is provided on one side of the inside of the body part 210 so as to mix the hydrogen peroxide vapor with air and discharge the hydrogen peroxide vapor mixed with air to the discharge part 250, so that the hydrogen peroxide vapor vaporized in the vaporization part 220 can move to a target point in the state of vapor, without being cold and thus liquefied while moving. This discharge fan 260 can be installed in any position as long as the hydrogen peroxide vapor vaporized in the vaporization part 220 can be smoothly mixed with air and discharged. However, it is preferable to install the discharge fan 260 on one side of a lower portion inside the body part 210. More preferably, the discharge fan 260 is installed at a relatively lower position than the vaporization part 220 and the heating part 240 inside the body part 210, so that the air supplied from the discharge part 600 can be heated to a certain temperature while passing through the heating part 240 and then mixed with the hydrogen peroxide vapor.

The air heater 261 may be further provided on one side of the inside of the body part 210, preferably between the discharge fan 260 and the vaporization part 220 so as to heat the air introduced through the discharge fan 260. The air heater 261 heats the air introduced through the discharge fan 260 to a certain temperature so that the air and the hydrogen peroxide vapor move upward easily inside the body part 210. Since vaporized hydrogen peroxide vapor is sensitive to temperature, the air heater 261 heats the air supplied by the discharge fan 260 so that the air is mixed with the hydrogen peroxide vapor in a state where the air is heated. Therefore, the hydrogen peroxide vapor can be completely diffused, without being condensed during diffusion. Such an air heater 261 may be any heating device that can heat the air supplied by the discharge fan 260 to a certain temperature, preferably about 50° C. to 70° C., and more preferably about 60° C.

The condensate tray 280 is placed on one inner side of the body part 210, preferably, between the air heater 261 and the vaporization part 220 so as to accommodate condensate that may form in at least one of the vaporization part 220, the heating part 240, the discharge part 250 and the diffusion fan 400, thereby preventing the condensate from flowing into the air heater 261 or the discharge fan 260 disposed at the lower portion of the body part 210. This condensate tray 280 is preferably formed with as large an area as possible without interfering with the flow of the air that is introduced through the discharge fan 260 and heated by the air heater 261. In this way, the condensate tray 280 is disposed at a predetermined distance from an upper portion of the air heater 261, so that the air heated by the air heater 261 can readily spread and rise inside the body part 210 through a lower surface of the condensate tray 280. Thus, spread air can be mixed with the hydrogen peroxide vapor more smoothly.

It would be obvious that the condensate tray 280, so named in the present invention, can also accommodate hydrogen peroxide solution that flows out or splashes out from the vaporization part 220, in addition to condensate.

The solution supply part 270 is provided on one side of the body part 210 or spaced apart from the body part 210 at a predetermined distance so as to supply hydrogen peroxide solution to the vaporization part 220. This solution supply part 270 includes a storage tank 271 for storing the hydrogen peroxide solution, a supply hose 273 connected to the storage tank 271, a supply nozzle 274 provided at one end of the supply hose 273, and a supply pump 272 for providing pressure so that the hydrogen peroxide solution can be supplied. Since the configuration of the solution supply part 270 is similar to those of general liquid supply devices, detailed description thereof will be omitted. At this time, an end portion of the supply nozzle 274 of the solution supply part 270 is preferably spaced apart from the bottom surface of the vaporization part 220 at an interval of about 10 mm to 20 mm, and more preferably about 15 mm. If such a separation length of the supply nozzle 274 is shorter than 10 mm, the hydrogen peroxide solution previously supplied to the vaporization part 220 and a distal end of the supply nozzle 274 may come into contact. To the contrary, if the separation length of the supply nozzle 274 exceeds 20 mm, there may be a problem in that the hydrogen peroxide solution splashes out of the vaporization part 220 due to increase of repulsive force generated when the hydrogen peroxide solution supplied from the supply nozzle 274 comes into contact with the bottom of the vaporization part 220. In addition, the solution supply part 270 preferably supplies the hydrogen peroxide solution at about 10 ml to 30 ml per minute, and preferably about 20 ml per minute. If a supply capacity of the hydrogen peroxide solution is less than 10 ml, energy efficiency decreases. If the supply capacity of the hydrogen peroxide solution exceeds 30 ml, the hydrogen peroxide solution cannot be easily vaporized since hydrogen peroxide solution is continuously supplied before the hydrogen peroxide solution supplied to the vaporization part 220 is sufficiently vaporized. As an amount of the hydrogen peroxide solution contained in the vaporization part 220 increases, energy efficiency of the heating part 240 may decrease. Therefore, in order to ensure that an appropriate amount of hydrogen peroxide solution is supplied to the inside of the vaporization part 220, it is preferable to adjust capacity of the hydrogen peroxide solution supplied from the solution supply part 270 appropriately within the range of 10 ml to 30 ml per minute.

The vapor decomposition device 300 is provided on one side of the inside of the housing 100, preferably on one side of the lower portion inside the housing 100 at a position relatively lower than that of the vaporizer 200 provided inside the housing 100 so as to decompose the hydrogen peroxide vapor diffused in a target space. It is possible to use any device as the vapor decomposition device 300 as long as it can smoothly decompose hydrogen peroxide vapor sprayed for sterilization inside the target space. According to an embodiment, the vapor decomposition device 300 may include vapor suction parts 310, a decomposition part 320 and a decomposed air discharge part 330.

The vapor suction parts 310 suctions the hydrogen peroxide vapor diffused inside the target space into the vapor decomposition device 300. At least one or more vapor suction parts 310 are provided on one side of the decomposition part 320, preferably, on one side of a lower portion of the decomposition part 320 so that the hydrogen peroxide vapor can be suctioned into the decomposition part 320 from the outside. Such a vapor suction part 310 may simply be formed of a through hole in one side of the lower portion of the decomposition part 320. However, in order to smoothly suction hydrogen peroxide vapor, it is preferable to provide a suction fan as the vapor suction part 310. Herein, it is preferable to further provide a filter at one side of the vapor suction part 310 so as to remove foreign substances such as dust contained in the hydrogen peroxide vapor while the external hydrogen peroxide vapor is suctioned.

The decomposition part 320 is to decompose the hydrogen peroxide vapor suctioned through the vapor suction parts 310. The decomposition part 320 can be any device that it can smoothly decompose the hydrogen peroxide vapor. According to an embodiment, a catalyst for decomposing the hydrogen peroxide vapor is provided inside the decomposition part 320 so that the hydrogen peroxide vapor can be decomposed. Herein, the catalyst may include a platinum Pt or palladium Pd catalyst, wherein in order to increase the decomposition efficiency of hydrogen peroxide vapor, the catalyst may further include a structure containing aluminum Al, silicon Si, etc. with other components, or at least one catalyst of iron oxide or nickel oxide. It is obvious that it is possible to use devices capable of decomposing hydrogen peroxide vapor through various methods in addition to the hydrogen peroxide vapor decomposition using a catalyst.

The decomposed air discharge part 330 is provided on one side of the decomposition part 320 so as to discharge air decomposed through the decomposition part 320 to the outside of the decomposition part 320. Such a decomposed air discharge part 330 is preferably provided on one side of the decomposition part 320, more preferably on one side of an upper portion of the decomposition part 320, and even more preferably on one side of an upper surface of the decomposition part 320. Since decomposed air that has been completely decomposed inside the decomposition part 320 has to be discharged from the inside of the decomposition part 320, the decomposed air discharge part 330 may be formed of at least one or more through holes, and may be further provided with a discharge fan to ensure smooth discharge depending on usage. In this way, the decomposed air discharged through the decomposed air discharge part 330 passes through the inside and outside of the vaporizer 200 that is provided above the housing 100 and flows into the diffusion fan 400.

The vapor suction parts 310 and the decomposed air discharge part 330 of the vapor decomposition device 300 are explained hereinabove with a focus on having a suction fan and a discharge fan, respectively. However, it is also possible to provide only one of a suction fan and a discharge fan depending on usage. In addition, when the hydrogen peroxide vaporization and decomposition apparatus 10 has a small capacity due to a small area of a target space, air may move using the discharge fan 260 of the vaporizer 200 instead of the suction fan and the discharge fan separately provided in the vapor decomposition device 300. In this way, it is possible to determine whether to provide a suction fan and a discharge fan respectively to the vapor suction parts 310 and the decomposed air discharge part 330 of the vapor decomposition device 300 in consideration of the area of a target space, the capacity of the hydrogen peroxide vaporization and decomposition apparatus 10, etc.

The vapor decomposition device 300 configured as described above is arranged such that a central axis thereof is offset from a central axis of the vaporizer 200, as shown in FIG. 3. Since the vapor decomposition device 300 and the vaporizer 200 are arranged so that the central axes thereof are offset from each other, the decomposed air discharged from the vapor decomposition device 300 collides with the vaporizer 200 so that turbulence is generated, which delays time for the decomposed air to be discharged to the outside. Accordingly, discharge speed of the decomposed air discharged from the vapor decomposition device 300 is also reduced due to internal pressure of the housing 100. Therefore, the hydrogen peroxide vapor stays longer inside the decomposition part 320 of the vapor decomposition device 300, which can increase the decomposition efficiency of the hydrogen peroxide vapor. In other words, the smoother the flow of the decomposed air introduced into the vapor decomposition device 300 so as to be discharged, the shorter the hydrogen peroxide vapor stays inside the decomposition part 320 of the vapor decomposition device 300. Therefore, in order to intentionally increase the time that the hydrogen peroxide vapor stays inside the decomposition part 320, the central axes of the vapor decomposition device 300 and the vaporizer 200 are arranged to be offset from each other so that the decomposed air discharged from the vapor decomposition device 300 forms turbulence inside the housing 100.

The diffusion fan 400 is provided on top of the housing 100, preferably, at one side on top of the housing 100 so as to be disposed spaced apart from the upper end of the discharge part 250 at a predetermined interval, and serves to diffuse the hydrogen peroxide vapor discharged from the discharge part 250 or air detoxified by a vapor detoxifier. This diffusion fan 400 may be any device that can smoothly diffuse the air decomposed in the vapor decomposition device 300 or discharged in the discharge part 250 of the vaporizer 200. Herein, a distance d between an introduction portion of the diffusion fan 400, through which air is introduced into the diffusion fan 400, and the discharge part 250 may be selectively determined according to capacity to be used. If the distance d between the introduction portion of the diffusion fan 400 and the discharge part 250 is long, a large amount of surrounding air is mixed with the hydrogen peroxide vapor so that before the hydrogen peroxide vapor is introduced into the diffusion fan 400, the hydrogen peroxide vapor may be condensed or some of the hydrogen peroxide vapor are discharged to the outside of the diffusion fan 400, thereby reducing diffusion efficiency. To the contrary, if the distance d between the introduction portion of the diffusion fan 400 and the discharge part 250 is short, suction force of the diffusion fan 400 is increased, so that the air supplied from the discharge fan 260 may be introduced into the diffusion fan 400 before being sufficiently heated, or the air may not be smoothly mixed with the hydrogen peroxide vapor, wherein the hydrogen peroxide solution splashed from the vaporization part 220 may be introduced into the diffusion fan 400 in a state where the hydrogen peroxide solution is not vaporized. Accordingly, the diffusion fan 400 is arranged so that the separation distance d from the discharge part 250 is preferably in the range of 10 mm to 150 mm, more preferably 30 mm to 70 mm, and even more preferably about 50 mm, considering the capacity to be used, surrounding environment, etc.

The discharge fan 260 has a capacity which is preferably determined according to the capacity of the diffusion fan 400. According to an embodiment, the discharge fan 260 has a capacity of 100 to 150 m3/h, and the diffusion fan 400 has a capacity of 500 to 1500 m3/h. More preferably, the discharge fan 260 has the capacity of about 120 m3/h, and the diffusion fan 400 has the capacity of about 1050 m3/h. In addition, an upper end area of the discharge part 250 is smaller than an area of the introduction portion of the diffusion fan 400 into which air is introduced. For example, if the introduction portion area of the diffusion fan 400 is 155 Φ, the upper end area of the discharge part 250 may be 100 Φ. The upper end area of the discharge part 250 is formed to be about 30% to 50% of the introduction portion area of the diffusion fan 400 and preferably about 40%, so that the hydrogen peroxide vapor discharged through the discharge part 250 is perfectly introduced into the diffusion fan 400, without leaking to the outside of the diffusion fan 400.

### Use of Hydrogen Peroxide Vaporization and Decomposition Apparatus

Modes of use of the hydrogen peroxide vaporization and decomposition apparatus 10 configured as above according to the present invention will be described in detail hereinafter.

### (Vaporization Process of Hydrogen Peroxide Vapor)

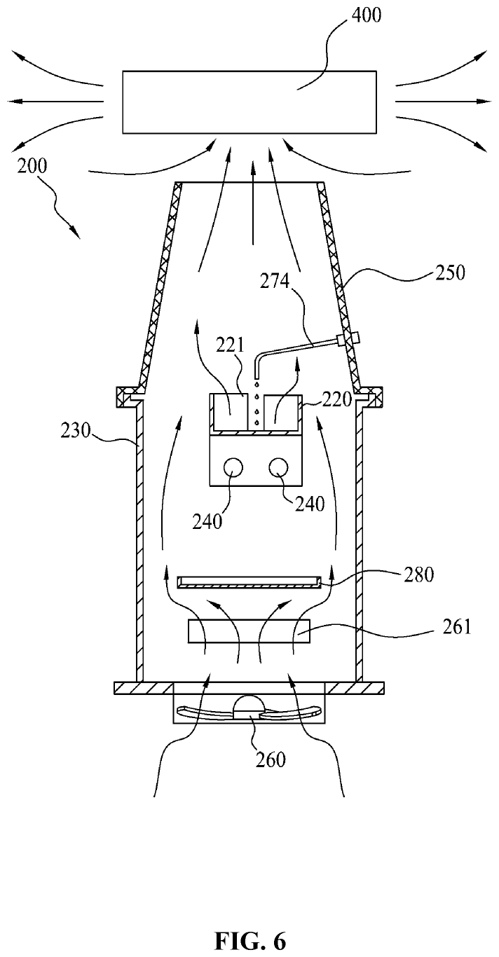

FIG. 6 is a cross-sectional view of the vaporizer according to the present invention, showing flows of fluid when vaporizing hydrogen peroxide vapor in the vaporizer 200.

First, the hydrogen peroxide vapor and decomposition device 10 is placed in a target space that requires sterilization.

Next, the vaporization part 220 is heated by means of the heating part 240 to a temperature at which the hydrogen peroxide solution may be vaporized. Herein, the heating part 240 includes two heater rods parallel to each other, as shown in FIG. 4, so that heat can be transferred uniformly to the entire vaporization part 220. Further, since the vaporization part 220 is fixed to the body part 210 at minimum contact points with respect to the body part 210 through the support parts 230, heat loss through the body part 210 can be minimized. Accordingly, the heat loss occurred when the heat supplied from the heating part 240 comes into contact with external air is minimized, thereby maximizing heating efficiency. In addition, the vaporization part 220 is disposed on the center of the body part 210 so as to be spaced apart from the body part 210 through the support parts 230 and to be separated from surrounding combustion materials, thereby improving stability of use even if a fire occurs in the vaporization part 220.

Next, the hydrogen peroxide solution is supplied from the solution supply part 270 to the vaporization part 220. The hydrogen peroxide solution is first supplied to the supply hose 273 connected to the storage tank 271. The supply nozzle 274 provided at a distal end of the supply hose 273 is disposed to supply the hydrogen peroxide solution to the vaporization part 220. Herein, an amount of the hydrogen peroxide solution supplied from the solution supply part 270 is appropriately controlled by controlling pressure of the supply pump 272 in consideration of capacity and vaporization speed of the vaporization part 220.

After that, the hydrogen peroxide solution supplied to the vaporization part 220 is vaporized in the vaporization part 220 heated through the heating part 240 so that hydrogen peroxide vapor is formed. Herein, even if the hydrogen peroxide solution supplied to the vaporization part 220 splashes from the bottom of the vaporization part 220 because of its drop, the splashed hydrogen peroxide solution comes into contact with the wall surfaces of the vaporization part 220 and is thus vaporized, thereby improving the vaporization efficiency. Further, if the vaporization part 220 has the protrusions or partition walls 210, the contact area of the hydrogen peroxide solution is increased owing to the protrusions or partition walls 210, thereby maximizing the vaporization efficiency of the hydrogen peroxide solution. As a result, the area of the vaporization part 220 can be reduced, thereby enabling the whole volumes of the hydrogen peroxide vaporizer 200 and the hydrogen peroxide vaporization and decomposition apparatus 10 to be decreased. Herein, the vaporization part 220 preferably vaporizes about 1 to 22 g of the hydrogen peroxide solution and more preferably about 8 to 20 g per minute.

Next, the hydrogen peroxide vapor generated from the vaporization part 220 is mixed with air by means of the discharge fan 260 and then discharged through the discharge part 250. Herein, the discharge part 250 is tapered upward so as to become gradually narrower toward the top, so that the hydrogen peroxide vapor vaporized in the vaporization part 220 may be smoothly mixed with the air supplied by the discharge fan 260, without arbitrarily scattering, and thus discharged through a discharge hole designated by a user. Further, the air introduced through the discharge fan 260 is heated to a temperature of about 60° C. while passing through the air heater 261, and primarily mixed with the hydrogen peroxide vapor vaporized in the vaporization part 220. In this way, the hydrogen peroxide vapor is discharged in the state where the hydrogen peroxide vapor is primarily mixed with high temperature air before being discharged through the discharge part 250.

Therefore, the temperature of the hydrogen peroxide vapor is not easily reduced while being diffused. As a result, the hydrogen peroxide vapor is smoothly diffused, without being condensed during diffusion. Moreover, the primary mixture stably can move in the upward direction of the discharge part 250, without flowing back, owing to the pressure of the discharge fan 260 and the heated air. At this time, since the air heated through the air heater 261 diffuses inside the body 210 along the lower surface of the condensate tray 280 disposed above the air heater 261, mixing with the hydrogen peroxide vapor vaporized in the vaporization part 220 can be achieved more smoothly.

Lastly, the primary mixture between the hydrogen peroxide vapor and the air discharged through the discharge parts 250, 500 is secondarily mixed with the surrounding air before being introduced into the diffusion fan 400 and then introduced into the diffusion fan 400. In this way, the hydrogen peroxide vapor and the air are mixed together through the secondary mixing and lower the concentration of hydrogen peroxide contained in the air, thereby enabling stable sterilization. The secondary mixture is diffused through the diffusion fan 400 and thus sterilizes a space, place, or article designated by the user. Herein, the discharge fan 260 and the diffusion fan 400 are set to have optimized diffusion capacities in consideration of a sterilization range, a vapor supply amount, or the like.

As described above, when the hydrogen peroxide vapor is continuously supplied, humidity inside the space to be sterilized increases. Therefore, even if the hydrogen peroxide vaporization and decomposition apparatus 10 according to the present invention is structurally optimized, condensation may occur at one side of the diffusion fan 400. However, the discharge part 250 according to the present invention is configured to gradually become narrower toward the top at an inclination angle a, so that most of the condensate generated from the diffusion fan 400 does not flow into the discharge part 250 but flows outward along a slope of the discharge part 250, and thus the introduction of the condensate is primarily blocked.

In addition, some of the condensate introduced into the discharge parts 250, 500 is collected by the condensate tray 280 provided inside the body part 210 so as to be prevented from being introduced into devices that use electricity, such as the air heater 261, the discharge fan 260 and the vapor decomposition device 300, and thus safe use is realized. Herein, the condensate tray 280 collects not only the condensate generated from the diffusion fan 400, the condensate generated from the vaporization part 220, the heating part 240 and the discharge part 250, and the hydrogen peroxide solution that splashes or leaks out of the vaporization part 220. Therefore, the hydrogen peroxide vaporizer 200 according to the present invention has a structure that allows safe use by preventing short circuits due to the condensate or the hydrogen peroxide solution.

### (Decomposition Process of Hydrogen Peroxide Vapor)

Hereinafter, process of decomposing the hydrogen peroxide vapor inside the target space after a predetermined time after diffusion of the hydrogen peroxide vapor into the target space through the process described above will be described in more detail with reference to the configuration described above.

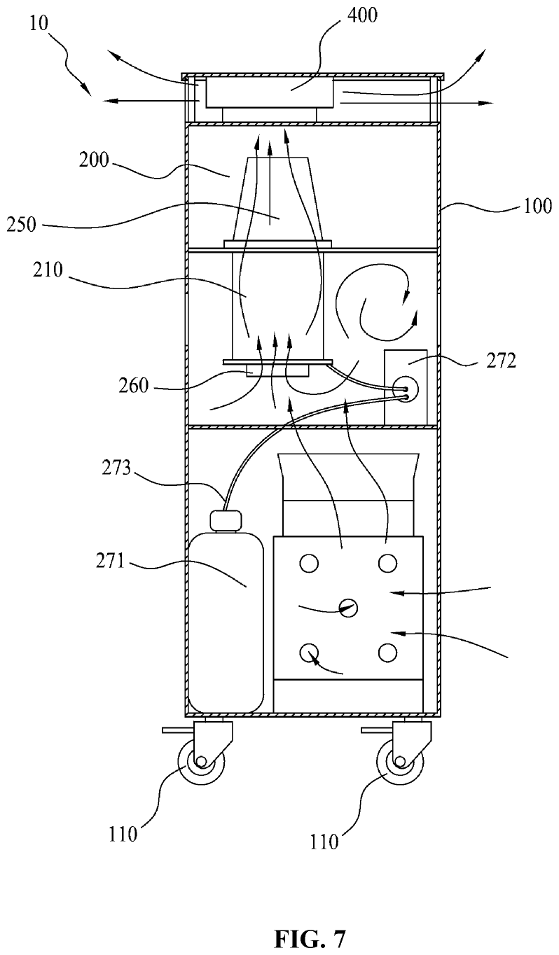

FIG. 7 is a longitudinal sectional view of the hydrogen peroxide vaporization and decomposition apparatus according to the present invention, showing flows of fluid according to the operation of the vapor decomposition device.

First, the hydrogen peroxide vapor diffused inside the target space is suctioned through the vapor suction parts 310 of the vapor decomposition device 300 and introduced into the decomposition part 320. Herein, as for the suction of the hydrogen peroxide vapor diffused inside the target space, the external hydrogen peroxide vapor can be suctioned using the suction force of at least any one of the suction fan provided on one side of the vapor suction parts 310, the discharge fan provided on one side of the vapor discharge part, the discharge fan 260 of the vaporizer 200, or the diffusion fan 400. In this way, in the suction of the hydrogen peroxide vapor diffused in the target space, external air can be suctioned using at least any one of the fan devices mentioned above according to the area of the target space, the capacity of the hydrogen peroxide vaporization and decomposition apparatus 10, etc.

Next, the hydrogen peroxide vapor is decomposed by a catalyst or the like for decomposing the hydrogen peroxide vapor inside the decomposition part 320. In the decomposition process, the hydrogen peroxide vapor can be decomposed through various methods depending on the type of decomposition part 320, wherein since these decomposition methods and processes are commonly used in the art, detailed descriptions thereof will be omitted.

Next, the decomposed air that has been completely decomposed in the decomposition part 320 is discharged through the decomposed air discharge part 330 formed on the upper surface of the decomposition part 320. Some of the decomposed air discharged in this way may be introduced into the vaporizer 200 disposed in the upper portion of the housing 100 through the upper surface of the decomposition part 320, and the remaining may be introduced into the diffusion fan 400 by passing through the outside of the vaporizer 200. However, in order to increase internal pressure by generating turbulence inside the housing 100 and improve the decomposition time of the hydrogen peroxide vapor, it is preferable to discharge the decomposed air only to the inside of the vaporizer 200. Herein, the discharge of the decomposed air discharged from the decomposition part 320 is also performed by at least one of the fan devices described above so that the decomposed air may naturally circulate.

In addition, the decomposed air discharged upward from the decomposition part 320 collides with the vaporizer 200, which is arranged so that the center thereof is offset from the center of the vapor decomposition device 300, so that turbulence may be formed at one side of the upper portion in the housing 100. In this way, since the centers of the vapor decomposition device 300 and the vaporizer 200 are arranged to be offset from each other, the air discharged from the vapor decomposition device 300 does not rise smoothly and delays the discharge of the decomposed air. Accordingly, the internal pressure of the housing 100 increases and the introduction of additional hydrogen peroxide vapor is also delayed. In other words, the time for the hydrogen peroxide vapor introduced into the decomposition part 320 to remain inside the decomposition part 320 increases, and the decomposition efficiency of the hydrogen peroxide vapor naturally improves.

Finally, the decomposed air discharged from the decomposition part 320 flows inside or outside the vaporizer 200 disposed in the upper portion of the housing 100 and is introduced into the diffusion fan 400. The decomposed air introduced into the diffusion fan 400 is discharged so as to be diffused into the target space through the diffusion fan 400.

The processes described above are continuously repeated for a certain period of time. When it is determined that the decomposition of the hydrogen peroxide vapor diffused inside the target space has been completed, the operation of the vapor decomposition device 300 can be stopped.

Herein, whether the decomposition of the hydrogen peroxide vapor inside the target space has been completed can be confirmed through various methods, which are commonly used in the corresponding industrial field. Therefore, there is no particular limitation on the methods of checking whether the decomposition of the hydrogen peroxide vapor is completed.

It could be understood by a person skilled in the art to which the present invention belongs will understand that the present invention as described above can be implemented in any other specific forms without changing the technical idea or essential features thereof. Therefore, the embodiments described hereinabove should be understood in all respects as illustrative and not restrictive. The scope of the present invention is indicated by the claims described later rather than the detailed description, and all changed or modified forms derived from the meaning and scope of the claims and the equivalent concept thereof should be construed as being included in the scope of the present invention.

### EXPLANATION OF REFERENCE SINGS

10: hydrogen peroxide vaporization and decomposition apparatus
100: housing
110: wheels
200: vaporizer
210: body part
220: vaporization part
221: partition walls
230: support parts
240: heating part
250: discharge part
260: discharge fan
261: air heater
270: solution supply part
271: storage tank
272: supply pump
273: supply hose
274: supply nozzle
280: condensate tray
300: vapor decomposition device
310: vapor suction parts
320: decomposition part
330: decomposed air discharge part
400: diffusion fan
a: inclination angle of discharge part
d: separation distance between diffusion fan introduction portion and discharge part

### ACKNOWLEDGEMENT

This work was supported by the Technology Development Program (S3129147) funded by the Ministry of SMEs and Startups (MSS, Korea).

1. A hydrogen peroxide vaporization and decomposition apparatus, comprising:
a housing;
a vaporizer provided on one side of the inside of the housing so as to vaporize a hydrogen peroxide solution;
a vapor decomposition device, which is provided on one side of the inside of the housing at a position relatively lower than that of the vaporizer so as to decompose hydrogen peroxide vapor, and of which a central axis is offset from a central axis of the vaporizer, wherein decomposed air discharged from the vapor decomposition device collides with the vaporizer and turbulence is generated, so that time for the decomposed air to be discharged to the outside is delayed and discharge speed of the decomposed air discharged from the vapor decomposition device is reduced due to internal pressure of the housing, thereby increasing time that the hydrogen peroxide vapor stays inside the vapor decomposition device and improving decomposition efficiency of the hydrogen peroxide vapor; and
a diffusion fan provided on an upper side of the housing so as to diffuse hydrogen peroxide vapor vaporized in the vaporizer or air decomposed in the vapor decomposition device,
wherein the vaporizer comprises:
a body part having the shape of a hollow column;
a vaporization part disposed on one side of the inside of the body part and having a predetermined capacity to accommodate the hydrogen peroxide solution;
support parts for supporting the vaporization part to allow the vaporization part to be spaced apart from the inside of the body part by a predetermined distance;
a heating part disposed on one side of the vaporization part to heat the vaporization part;
a discharge part disposed on top of the body part to induce the hydrogen peroxide vapor generated from the vaporization part to be discharged;
a discharge fan provided so as to be disposed on one side of the inside of the body part at a position relatively lower than that of the vaporization part, thereby improving discharge efficiency of the hydrogen peroxide vapor that is vaporized in the vaporization part and discharged through the discharge part; and
a condensate tray, which is provided between the vaporization part and the discharge fan so as to accommodate condensate generated from at least one of the vaporization part, the heating part, the discharge part, and the diffusion fan, and which is disposed above the discharge fan so as to allow air supplied through the discharge fan along a lower surface of the condensate tray to spread inside the body part and to mix more smoothly with the hydrogen peroxide vapor vaporized in the vaporization part.

2. The hydrogen peroxide vaporization and decomposition apparatus according to claim 1, wherein the diffusion fan is provided so as to be spaced apart from an upper end of the discharge part at an interval in the range of 10 mm to 150 mm.

3. The hydrogen peroxide vaporization and decomposition apparatus according to claim 1, wherein the vaporization part is configured so that a distance from a vapor generation surface of the vaporization part to the upper end of the discharge part has a height in the range of 50 mm to 300 mm.

4. The hydrogen peroxide vaporization and decomposition apparatus according to claim 1, wherein the upper end area of the discharge part is formed to be 30% to 50% of an introduction portion area of the diffusion fan into which the hydrogen peroxide vapor or the air decomposed in the vapor decomposition device is introduced.
